(19) United States
(12) Patent Application Publication
(10) Pub. No.: US 2017/0027828 A1
(43) Pub. Date: Feb. 2, 2017

(54) SUN PROTECTION COMPOSITION AND APPLICATION THE SAME

(71) Applicant: National Tsing Hua University, Hsinchu City (TW)

(72) Inventors: Chi-Young Lee, Hsinchu County (TW); Min-Chiao Tsai, Tainan City (TW)

(21) Appl. No.: 15/082,800

(22) Filed: Mar. 28, 2016

(30) Foreign Application Priority Data
Jul. 31, 2015 (TW) 104124921

(51) Int. Cl. A61K 8/29; A61K 8/37; A61K 8/40; D06M 13/127; A61K 8/35; A61K 8/46; D06M 13/224; D06M 11/46; A61Q 17/04; A61K 8/365

(52) U.S. Cl. CPC A61K 8/29; A61Q 17/04; A61K 8/37; A61K 8/40; A61K 8/365; A61K 8/35; A61K 8/466; D06M 13/2246; D06M 11/46; D06M 13/127; A61K 2800/413

(57) ABSTRACT

Provided is a sun protection composition including a UV absorber and a plurality of porous titanium dioxide microspheres. The UV absorber absorbs light of at least one of UVA radiation and UVB radiation. The particle size of the porous titanium dioxide microspheres is 100 nm to 300 nm, and the porous titanium dioxide microspheres can scatter light in a wavelength range between 200 nm and 400 nm. A cosmetic and a fabric containing the sun protection composition can also scatter light in a wavelength range between 200 nm and 400 nm, such that the UV protection capability of the cosmetic and the fabric is enhanced.

### CROSS-REFERENCE TO RELATED APPLICATION

This application claims the priority benefit of Taiwan application serial no. 104124921, filed on Jul. 31, 2015. The entirety of the above-mentioned patent application is hereby incorporated by reference herein and made a part of this specification.

### BACKGROUND OF THE INVENTION

Field of the Invention

The invention relates to a sun protection composition, and more particularly, to a cosmetic and a fabric containing the sun protection composition.

Description of Related Art

UV causes damages such as sun burn and sun tan to the human skin, and can even cause, for instance, skin cancer. UV in sunlight can be categorized into three groups according to the wavelength: long-wave UV (UVA), medium-wave ultraviolet (UVB), and short-wave UV (UVC). In particular, UVA has very strong penetration power, and can reach the dermal layer of the skin, thus facilitating aging of the skin, even causing skin cancer. UVA causes chronic and long-term damage to the skin, and since UVA has the highest proportion in the components of UV in sunlight, sun protection is even more important.

Due to increased demand for sun protection, the medical and beauty industries have flourished in recent years. In general, sun protection principles are divided into two broad categories: physical sun protection and chemical sun protection. Physical sun protection blocks UV with the principle of using a sunscreen to reflect or scatter light. In chemical sun protection, UV is absorbed by using a chemical substance to convert the chemical substance into molecular vibrational energy or heat energy to eliminate UV damage.

Although many international manufacturers continuously develop new sun protection products, the sun protection efficacy of physical sun protection cannot be significantly enhanced. Both the market and the industry emphasize the nanonization of a physical sunscreen to prevent excessively white makeup; however, the nanonization of the physical sunscreen cannot provide better UV protection capability, and instead the usage amount of the physical sunscreen or the chemical sunscreen needs to be increased. However, the nanoparticles in the physical sunscreen may increase the difficulty of dispersion of a powder in an emulsion, and may also cause the potential risk of being inhaled into the body; and an increase in the chemical sunscreen causes damage to the skin.

### SUMMARY OF THE INVENTION

The invention provides a sun protection composition and a cosmetic and a fabric containing the sun protection composition capable of scattering light in a wavelength range between 200 nm and 400 nm, such that the UV protection capability of the cosmetic and the fabric is enhanced.

The invention provides a sun protection composition including a UV absorber and a plurality of porous titanium dioxide microspheres. The UV absorber absorbs light of at least one of UVA radiation and UVB radiation. The particle size of the porous titanium dioxide microspheres is 100 nm to 300 nm, and the porous titanium dioxide microspheres can scatter light in a wavelength range between 200 nm and 400 nm.

In an embodiment of the invention, based on 100 wt % of the sun protection composition, the content of the porous titanium dioxide microspheres is 1 wt % to 15 wt % and the content of the UV absorber is less than 15 wt %.

In an embodiment of the invention, the UV absorber includes: a component (A), a component (B), or a combination thereof The component (A) includes: avobenzone, oxybenzone, terephthalylidene dicamphor sulfonic acid, or a combination thereof The component (B) includes: octyl methoxycinnamate, octocrylene, salicylate, or a combination thereof

In an embodiment of the invention, based on 100 wt % of the UV absorber, the content of the component (A) is less than 15 wt % and the content of the component (B) is less than 15 wt %.

hi an embodiment of the invention, the ratio of the long diameter and the short diameter of every porous titanium dioxide microsphere is between 0.5 and 1.5.

In an embodiment of the invention, the difference of any two diameters of the porous titanium dioxide microspheres is less than 50 nm.

In an embodiment of the invention, the particle size distribution of the porous titanium dioxide microspheres is less than 20%.

In an embodiment of the invention, the sun protection composition further includes a plurality of ultrafine titanium dioxide spheres. The particle size of the ultrafine titanium dioxide spheres is less than the particle size of the porous titanium dioxide microspheres.

In an embodiment of the invention, the ultrafine titanium dioxide spheres, the porous titanium dioxide microspheres, and the UV absorber are mixed to form a single agent.

The invention provides a sun protection set including the sun protection composition. The porous titanium dioxide microspheres and the UV absorber are mixed to form a first agent. The ultrafine titanium dioxide spheres with absorber are formed into a second agent.

A method of using the sun protection set includes the following steps. A first sun protection layer is formed by using the first agent. A second sun protection layer is formed by using the second agent to cover the first sun protection layer.

In an embodiment of the invention, the second sun protection layer includes one sun protection layer, two sun protection layers, or a plurality of sun protection layers.

The invention provides a cosmetic having sun protection efficacy including the sun protection composition. The cosmetic is an emulsion, a cream, a suspension, a gel, a powder, or a combination thereof.

In an embodiment of the invention, when the cosmetic is an emulsion, a cream, a suspension, or a gel, the cosmetic further includes a carrier oil, an emulsifier, an antibacterial agent, a humectant, and a solvent.

The invention provides a fabric having sun protection efficacy including the sun protection composition. The sun protection composition covers the surface of a substrate or is mixed in the substrate.

Based on the above, the sun protection composition of the invention has porous titanium dioxide microspheres having a particle size of 100 nm to 300 nm, such that the sun protection composition can scatter light in a wavelength range between 200 nm and 400 nm. Therefore, the cosmetic and the fabric of the invention containing the sun protection composition can scatter light in a wavelength range between 200 nm and 400 nm, such that the UV protection capability of the cosmetic and the fabric is enhanced.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is an electron micrograph of porous titanium dioxide microspheres.

FIG. 2A to FIG. 2E are respectively various stacked structures of porous titanium dioxide microspheres and ultrafine titanium dioxide spheres of embodiments of the invention.

FIG. 3A is the UV-visible light absorption spectra of experimental example 1 and comparative examples 1 to 3.

FIG. 3B is the UV-visible light absorption spectra of experimental example 2 and comparative examples 4 to 5.

FIG. 4A is the UV-visible light absorption spectra of experimental examples 2 to 3 and comparative examples 6 to 8.

FIG. 4B is the UV-visible light absorption spectra of comparative examples 9 to 11.

FIG. 5 is the absorption spectra of UV test papers of experimental examples 4 to 7 and comparative example 12.

FIG. 6A is the absorption spectra of UV test papers of experimental example 4a, experimental example 8, and comparative example 13 after 2 minutes of continuous UV irradiation.

FIG. 6B is the absorption spectra of UV test papers of experimental example 4a, experimental example 8, and comparative example 13 with leaving the test papers for 20 minutes prior to 2 minutes of continuous UV irradiation.

### DESCRIPTION OF THE EMBODIMENTS

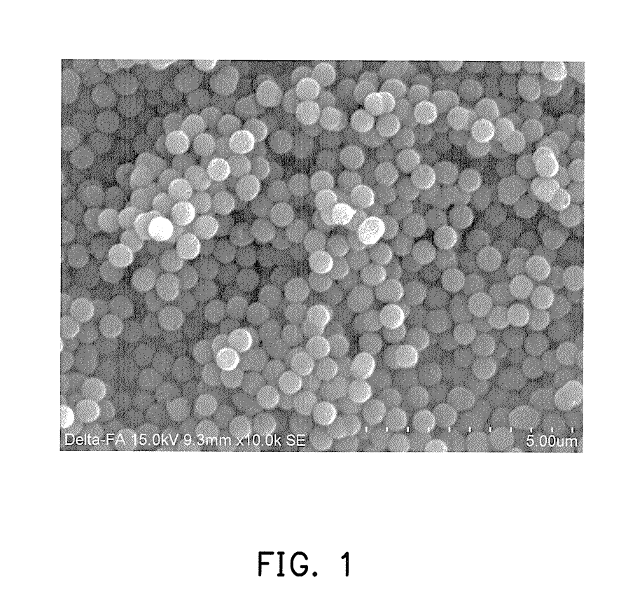

FIG. 1 is an electron micrograph of porous titanium dioxide microspheres.

The invention provides a sun protection composition including a UV absorber and a plurality of porous titanium dioxide microspheres. The content of the UV absorber is less than 15 wt %. The content of the porous titanium dioxide microspheres is 1 wt % to 15 wt %. The contents described here refer to weight percentages of each component to the overall sun protection composition.

The UV absorber includes: a component (A), a component (B), or a combination thereof In the present embodiment, the component (A) includes:

avobenzone, oxybenzone, terephthalylidene dicamphor sulfonic acid (Mexoryl SX), or a combination thereof; and the component (B) includes: octyl methoxycinnamate, octocrylene, salicylate, or a combination thereof In other embodiments, the UV absorber is not particularly limited, provided the UV absorber can absorb light of at least one of UVA and UVB wave bands (radiation), and the invention is not limited thereto. In general, the UVA wavelength is about 315 nm to about 400 nm, and the UVB wavelength is about 280 nm to about 315 nm. In an embodiment, the content of the component (A) is 0 wt % to 15 wt % of the content of the UV absorber. The content of the component (B) is 0 wt % to 15 wt % of the content of the UV absorber.

Referring to FIG. 1, the shape of the porous titanium dioxide microspheres of an embodiment of the invention is a circle, such as a perfect circle or a rough circle, and the size is uniform. The particle size of the plurality of porous titanium dioxide microspheres is, for instance, 100 nm to 300 nm. The circle or the rough circle here implies that the ratio of the long diameter and the short diameter of every porous titanium dioxide microsphere is substantially close to 1. In an embodiment, the ratio of the long diameter and the short diameter of every porous titanium dioxide microsphere is 0.5 to 1.5. In an embodiment, the ratio of the long diameter and the short diameter of every porous titanium dioxide microsphere is 0.8 to 1.2. In another embodiment, the difference of any two diameters of porous titanium dioxide microspheres is less than 50 nm. In another embodiment, the particle size distribution of the plurality of porous titanium dioxide microspheres is less than 20%. The porous titanium dioxide microspheres can scatter light in a wavelength range between 200 nm and 400 nm. The scattering properties allow the sun protection composition of the invention to have high absorption capability for UV wave bands such that the sun protection composition can effectively improve the existing physical and chemical sunscreen, and thereby improve UV blocking capability. In the invention, porous titanium dioxide microspheres having uniform size and good dispersibility (monodispersed) can be synthesized in an autoclave by a self-sacrificing template method. Under suitable parameter regulations, the particle size of the porous titanium dioxide microspheres can be adjusted. By adjusting the particle size of the porous titanium dioxide microspheres, the UV-visible light absorption spectrum thereof can be further regulated.

When the particle size of the porous titanium dioxide microspheres (T-PRO) of the invention is less than 300 nm, strong absorption occurs at UVB (280 nm to 315 nm) and UVA (315 nm to 400 nm) wave bands. Therefore, the porous titanium dioxide microspheres of the invention have better protection capability against UVA and UVB readily causing skin tan, sun burn, or even skin cancer.

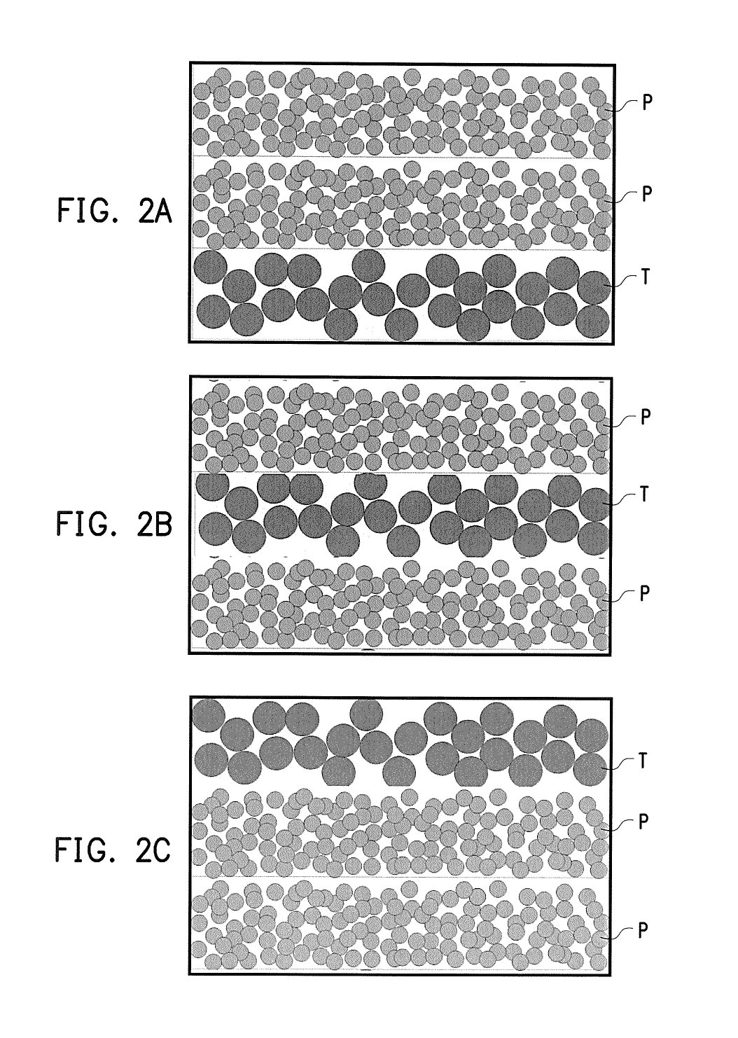

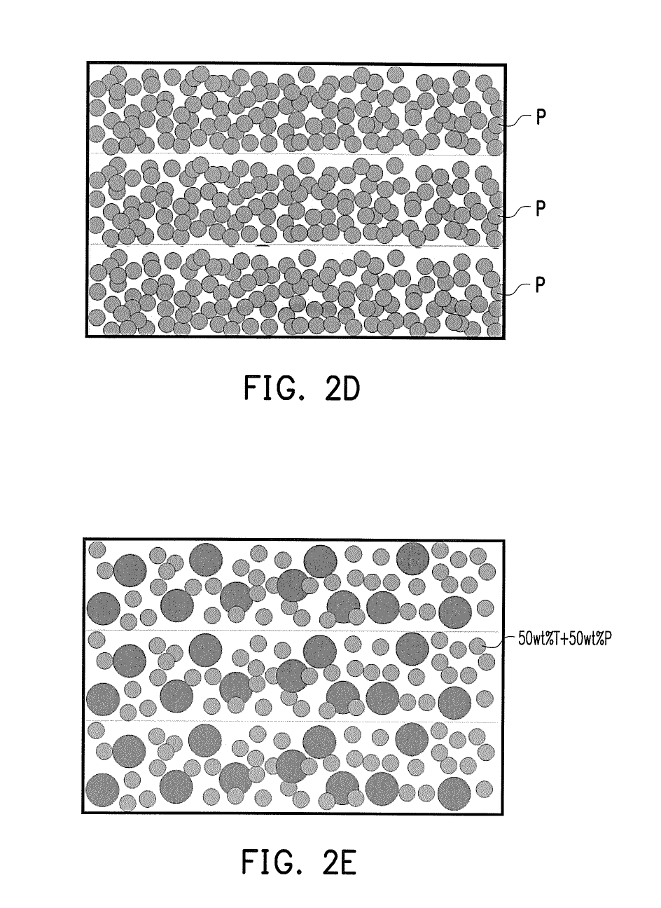

FIG. 2A to FIG. 2E are respectively various stacked structures of porous titanium dioxide microspheres and ultrafine titanium dioxide spheres of embodiments of the invention.

In another embodiment, the sun protection composition can be used with various commercial sun protection products to enhance the UV protection capability thereof. More specifically, the porous titanium dioxide microspheres and the UV absorber can be mixed to form a first agent; and the ultrafine titanium dioxide spheres (i.e., commercial sun protection product) can be used as a second agent. The method in which various commercial sun protection products are used can adopt the several embodiments in the following. In an embodiment, a first sun protection layer is first formed by using the first agent. Then, a second sun protection layer is formed by using the second agent to cover the first sun protection layer. In some embodiments, the second sun protection layer can be, for instance, one sun protection layer, two sun protection layers, or a plurality of sun protection layers. In other words, the first sun protection layer (including the porous titanium dioxide microspheres of the present embodiment) is used as a bottom layer, and the second sun protection layer (including a commercial sun protection product) covering the first sun protection layer can be coated according to a user's needs. For instance, as shown in FIG. 2A, a titanium dioxide thin film T is first foinied by using porous titanium dioxide microspheres, and then two ultrafine titanium dioxide sphere thin films P are respectively formed on the titanium dioxide thin film T by using ultrafine titanium dioxide spheres. As a result, a titanium dioxide stacked structure TPP shown in FIG. 2A can be formed. In another embodiment, as shown in FIG. 2B, an ultrafine titanium dioxide sphere thin film P is first foil ied by using ultrafine titanium dioxide spheres, and then a titanium dioxide thin film T is formed on the ultrafine titanium dioxide sphere thin film P. Then, another ultrafine titanium dioxide sphere thin film P is formed on the titanium dioxide thin film T. As a result, a titanium dioxide stacked structure PTP shown in FIG. 2B can be formed. In other embodiments, as shown in FIG. 2C, two ultrafine titanium dioxide sphere thin films P are first respectively formed by using ultrafine titanium dioxide spheres, and then a titanium dioxide thin film T is formed on the two ultrafine titanium dioxide sphere thin films P. As a result, a titanium dioxide stacked structure PPT shown in FIG. 2C can be formed. In other embodiments, as shown in FIG. 2D, three ultrafine titanium dioxide sphere thin films P are respectively formed by using ultrafine titanium dioxide spheres to form a titanium dioxide stacked structure PPP shown in FIG. 2D.

Moreover, the ultrafine titanium dioxide spheres, the porous titanium dioxide microspheres, and the UV absorber can also be mixed to form a single agent. During use, the single agent can be directly coated on a surface of a target, and the formed sun protection thin film is as shown in FIG. 2E. The absorption spectra of the UV test papers of the various stacked structures of FIG. 2A to FIG. 2E are described in detail later in the specification and are therefore not described here.

Not only can the sun protection composition of the invention be used as a single sun protection product, the sun protection composition can also be applied in a cosmetic such that the cosmetic has sun protection efficacy. In addition to the sun protection composition, the cosmetic further includes a carrier oil, an emulsifier, an antibacterial agent, a humectant, and a solvent. In an embodiment, the content of the carrier oil can be, for instance, 18 wt % to 22 wt %. The content of the emulsifier can be, for instance, 1.8 wt % to 2.2 wt %. The content of the antibacterial agent can be, for instance, 0 wt % to 1 wt %. The content of the humectant can be, for instance, 9 wt % to 11 wt %. The content of the solvent can be, for instance, 50 wt % to 65 wt %. However, the invention is not limited thereto. The contents described here refer to weight percentages of each component to the overall cosmetic. In an embodiment, the cosmetic having sun protection efficacy of the invention can be, for instance, an emulsion, a cream, a suspension, a gel, a powder, or a combination thereof

Moreover, the sun protection composition of the invention can also be applied in a fabric, such that the fabric has sun protection efficacy. The fabric having sun protection efficacy includes covering a surface of a substrate with the sun protection composition or mixing the sun protection composition in the substrate. Although the present embodiment is exemplified by a fabric, since the sun protection composition has strong absorption capability at UVB and UVA wave bands, the invention can be applied in various different substrates to form various products having sun protection efficacy. For instance, the substrate can be glass, transparent plastic, an umbrella, a fabric, or a substrate of various products requiring sun protection efficacy, and the invention does not particularly limit the application scope of the sun protection composition. To apply the sun protection composition to different substrates, those skilled in the art should know that, the sun protection composition can further include an additive such as a smooth softener, a crosslinking agent, an adhesive, and a thickener, and different contents can be adopted according to the designer's needs to apply the sun protection composition to the substrate of different products.

A plurality of experimental examples is provided below to further describe the sun protection composition of the invention and the cosmetic and the fabric containing the sun protection composition. In the following, the degree of absorption for a wavelength of 200 nm to 800 nm was tested with a NanoDrop spectrophotometer (made by J&H Technology Co., Ltd.)

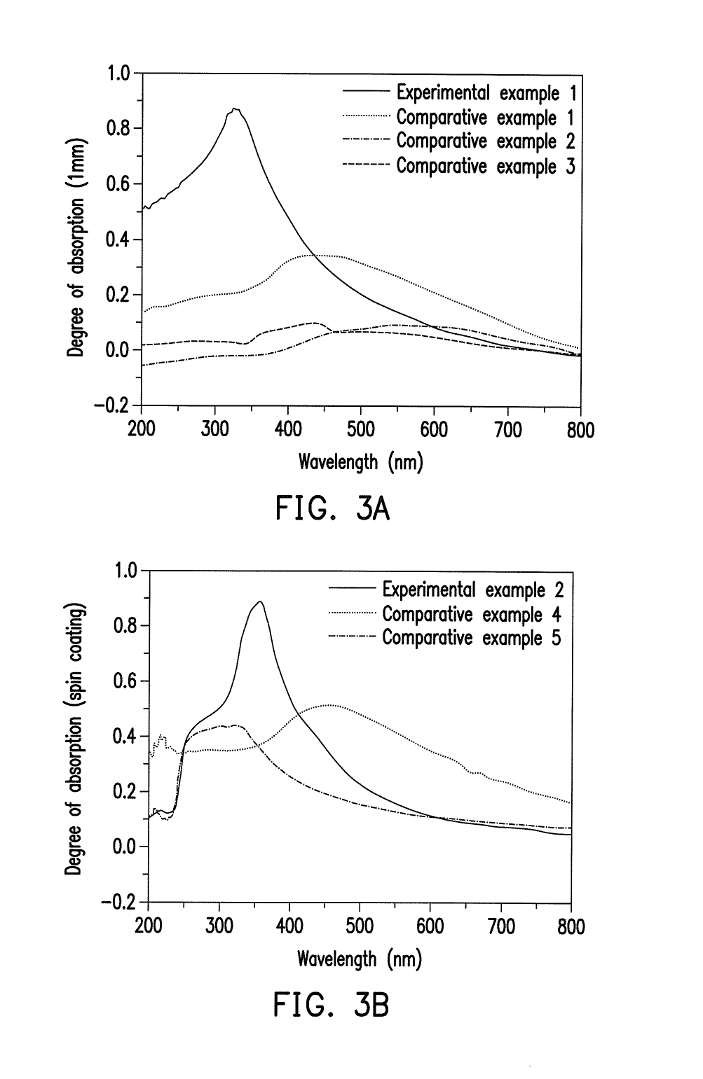

FIG. 3A is the UV-visible light absorption spectra of experimental example 1 and comparative examples 1 to 3. FIG. 3B is the UV-visible light absorption spectra of experimental example 2 and comparative examples 4 to 5.

### EXPERIMENTAL EXAMPLE 1

In experimental example 1, porous titanium dioxide microspheres having a particle size of 200 nm to 250 nm were synthesized in an autoclave by using a self-sacrificing template method. Then, the porous titanium dioxide microspheres having a concentration of 0.02 wt % were placed in a quartz cuvette having a light transmission path of 1 mm, and a UV-visible light absorption spectrum test was performed by using an ultramicro spectrophotometer. The results thereof are as shown in FIG. 3A.

### COMPARATIVE EMAMPLES 1 TO 3

The difference between comparative examples 1 to 3 and experimental example 1 is that in comparative examples 1 to 3, different commercial titanium dioxides were respectively used to perform a UV-visible light absorption spectrum test, and the test method thereof is the same as that of experimental example 1. Specifically, in comparative example 1, a titanium dioxide pigment (product of DuPont, model: R102) having a concentration of 0.02 wt % was used. In comparative example 2, a titanium dioxide pigment (product of DuPont, model: R706) having a concentration of 0.02 wt % was used. In comparative example 3, titanium dioxide (made by Sigma-Aldrich Corporation) having a concentration of 0.02 wt % and a particle size of 5 nm was used. UV-visible light absorption spectrum tests were respectively performed by using the ultramicro spectrophotometer. The results are as shown in FIG. 3A.

The results of FIG. 3A show that, the degree of absorption of the porous titanium dioxide microspheres having a particle size of 200 nm to 250 nm of experimental example 1 at UVB (280 nm to 315 nm) and UVA (315 nm to 400 nm) wave bands is much greater than that of the commercial titanium dioxides of comparative examples 1 to 3. In other words, the porous titanium dioxide microspheres of experimental example 1 have better protection capability against UV at UVA and UVB wave bands.

### EXPERIMENTAL EXAMPLE 2

The sample of experimental example 2 was porous titanium dioxide microspheres having a particle size of 200 nm to 250 nm synthesized in an autoclave by using a self-sacrificing template method. Then, the porous titanium dioxide microspheres having a concentration of 1 wt % were coated on a transparent substrate by using a spin coating method to form a titanium dioxide thin film. Then, a UV-visible light absorption spectrum test was performed by using an ultramicro spectrophotometer.

The results thereof are as shown in FIG. 3B.

### COMPARATIVE EXAMPLES 4 TO 5

The difference between comparative examples 4 to 5 and experimental example 2 is that various commercial titanium dioxides were used for the samples of comparative examples 4 to 5. Specifically, a titanium dioxide pigment (product of DuPont, model: R102) having a concentration of 1 wt % was used for the sample of comparative example 4. The sample of comparative example 5 was a photocatalyst (made by Evonik Industries, model: P25) having a concentration of 1 wt %, and the results thereof are as shown in FIG. 3B.

The results of FIG. 3B show that, the degree of absorption of the porous titanium dioxide microspheres having a particle size of 200 nm to 250 nm of experimental example 2 at UVB (280 nm to 315 nm) and UVA (315 nm to 400 nm) wave bands is much greater than that of the commercial titanium dioxides of comparative example 4 and comparative example 5. In other words, experimental example 2 has better protection capability against UV at UVA and UVB wave bands.

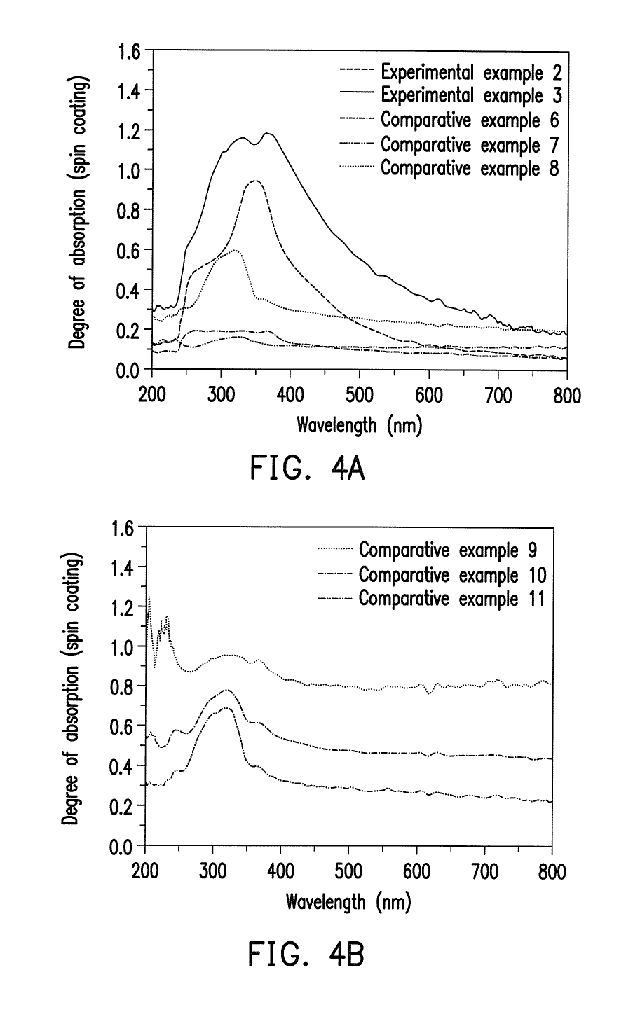

FIG. 4A is the UV-visible light absorption spectra of experimental examples 2 to 3 and comparative examples 6 to 8. FIG. 4B is the UV-visible light absorption spectra of comparative examples 9 to 11.

### EXPERIMENTAL EXAMPLE 3

The difference between experimental example 3 and experimental example 2 is that, in addition to the 1 wt % of porous titanium dioxide microspheres of experimental example 2, 2.5 wt % of UVB absorber-octyl methoxycinnamate was also added in the sample of experimental example 3. The results are as shown in FIG. 4A.

### COMPARATIVE EXAMPLES 6 TO 11

The difference between comparative examples 6 to 11 and experimental example 3 is that the samples of comparative examples 6 and 8 to 11 adopt various commercial zinc oxides respectively containing or not containing a UVB absorber. The results thereof are as shown in FIG. 4A and FIG. 4B. Specifically, the sample used in comparative example 6 was 1 wt % of zinc oxide. The sample used in comparative example 7 was 2.5 wt % of octyl methoxycinnamate. The sample used in comparative example 8 was formed by adding 2.5 wt % of octyl methoxycinnamate to 1 wt % of zinc oxide. The sample used in comparative example 9 was formed by adding 2.5 wt % of octyl methoxycinnamate to 5.0 wt % of zinc oxide. The sample used in comparative example 10 was formed by adding 2.5 wt % of octyl methoxycinnamate to 2.5 wt % of zinc oxide. The sample used in comparative example 11 was formed by adding 2.5 wt % of octyl methoxycinnamate to 1.0 wt % of zinc oxide, and the results thereof are as shown in FIG. 4B. Basically, comparative example 8 and comparative example 11 have the same compositions, but the degrees of absorption of the two are normalized results, and therefore are not exactly the same. It should be mentioned that, the zinc oxides used in comparative example 6 and comparative examples 8 to 11 were all separated from the commercial CHANEL UV ESSENTIEL.

The results of FIG. 4A and FIG. 4B show that, at UVB (280 nm to 315 nm) and UVA (315 nm to 400 nm) wave bands, the degree of absorption of the porous titanium dioxide microspheres having a particle size of 200 nm to 250 nm of experimental example 3 (including 2.5 wt % of octyl methoxycinnamate) is much greater than the degrees of absorption of comparative examples 6 to 11, and is even better than the degree of absorption of the porous titanium dioxide microspheres having a particle size of 200 nm to 250 nm of experimental example 2 (without 2.5 wt % of octyl methoxycinnamate). Therefore, after 2.5 wt % of octyl methoxycinnamate was added to porous titanium dioxide microspheres having a particle size of 200 nm to 250 nm, the UV protection capability for UVA and UVB wave bands is better.

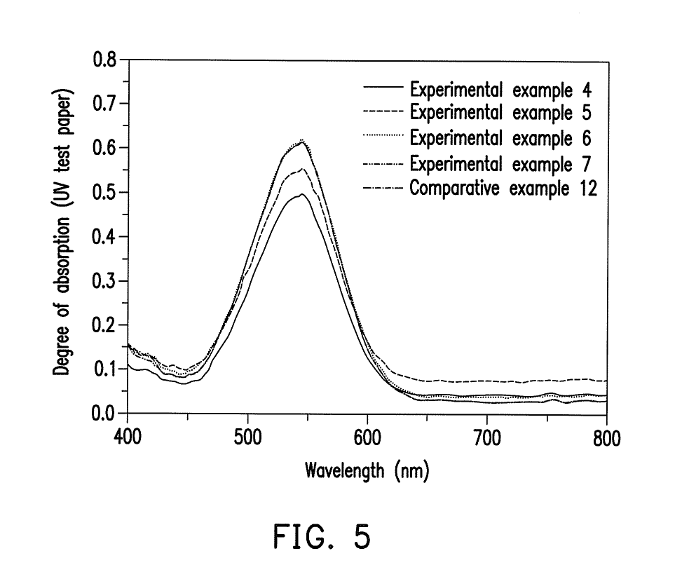

FIG. 5 is the absorption spectra of UV test papers of experimental examples 4 to 7 and comparative example 12.

### EXPERIMENTAL EXAMPLE 4

A titanium dioxide stacked structure TPP shown in FIG. 2A was formed. The forming method includes coating porous titanium dioxide microspheres having a concentration of 1 wt % and a particle size of 200 nm to 250 nm with octyl methoxycinnamate on UV test papers using a spin coating method to form a titanium dioxide thin film T. Then, 1 wt % of ultrafine titanium dioxide spheres were respectively coated on the porous titanium dioxide microspheres using a spin coating method to form two ultrafine titanium dioxide sphere thin films P. Then, the UV test papers were continuously irradiated by UV for 1 minute to measure the degree of coloration of each UV test paper. The results thereof are as shown in FIG. 5.

### EXPERIMENTAL EXAMPLES 5 TO 6

The method of experimental examples 5 to 6 is similar to that of experimental example 4, and the difference is that the formed structures are respectively the titanium dioxide stacked structures PTP and PPT of FIG. 2B and FIG. 2C. The results thereof are as shown in FIG. 5.

### COMPARATIVE EXAMPLE 12

The method of comparative example 12 is similar to that of experimental example 4, and the difference is that the formed structure is the titanium dioxide stacked structure PPP of FIG. 2D. The results thereof are as shown in FIG. 5.

### EXPERIMENTAL EXAMPLE 7

The titanium dioxide structure 50 wt % T+50 wt % P as shown in FIG. 2E was formed. The forming method includes adding 50 wt % of ultrafine titanium dioxide spheres in 50 wt % of 200 nm to 250 nm porous titanium dioxide microspheres, and then coating the mixture on a UV test paper with a spin coating method. The results are as shown in FIG. 5.

For the UV test paper test, the greater the degree of coloration, the worse the effect of UV absorption or blocking by the titanium dioxide stacked structure on the UV test paper. The results of FIG. 5 show that, the degree of coloration of the titanium dioxide stacked structure TPP of experimental example 4 is less, and the effect of UV absorption or blocking thereof is better. In comparison to the titanium dioxide stacked structures PTP (experimental example 5), PPT (experimental example 6), and PPP (experimental example 12) and the titanium dioxide structure 50 wt % T+50 wt % P (experimental example 7), the titanium dioxide stacked structure TPP of experimental example 4 has greater UV protection capability, and can provide an additional 15% UV protection capability.

Moreover, for different titanium dioxide stacked structures, the effect of UV blocking of the titanium dioxide stacked structure TPP of experimental example 4 is greater than the effect of UV blocking of the titanium dioxide stacked structure PTP of experimental example 5. The effect of UV blocking of the titanium dioxide stacked structure PTP of experimental example 5 is greater than the effect of UV blocking of the titanium dioxide stacked structure PPT of experimental example 6. In other words, the porous titanium dioxide microspheres of the invention were used as a bottom layer, such that the effect of UV absorption of a physical sunscreen can be enhanced.

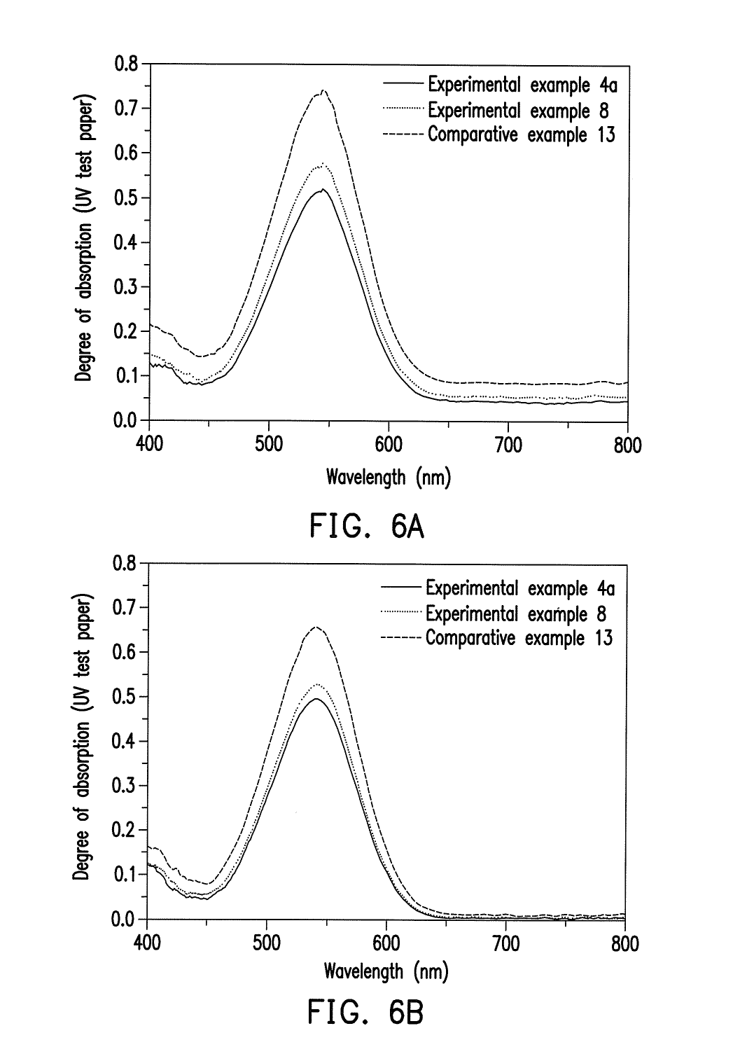

FIG. 6A is the absorption spectra of UV test papers of experimental example 4a, experimental example 8, and comparative example 13 after 2 minutes of continuous UV irradiation. FIG. 6B is the absorption spectra of UV test papers of experimental example 4a, experimental example 8, and comparative example 13 with leaving the test sample for 20 minutes prior to 2 minutes of continuous UV irradiation.

### EXPERIMENTAL EXAMPLE 4a

The forming method of experimental example 4a is similar to that of experimental example 4, and the difference thereof is that a UVA absorber was added in the titanium dioxide stacked structure TPP formed in experimental example 4a. Specifically, porous titanium dioxide microspheres having a concentration of 1 wt % and a particle size of 200 nm to 250 nm were added in 2 wt % of 2-hydroxy-4-methoxybenzophenone, wherein 2-hydroxy-4-methoxybenzophenone is the UVA absorber. Then, the mixture was coated on an UV test paper using a spin coating method to form a titanium dioxide thin film T. Then, after 2 wt % of 2-hydroxy-4-methoxybenzophenone was added in titanium dioxide having a concentration of 1 wt % (made by Sigma-Aldrich), the mixture was coated on the porous titanium dioxide microspheres using a spin coating method to form two titanium dioxide sphere thin films P. Then, the UV test paper was continuously irradiated by UV for 1 minute to measure the degree of coloration of the UV test paper. The results thereof are as shown in FIG. 6A. The UV test paper was left to stand for 20 minutes prior to 2 minutes of continuous UV irradiation, then measure the absorption spectra thereof. The results thereof are as shown in FIG. 6B.

### EXPERIMENTAL EXAMPLE 8

In experimental example 8, porous titanium dioxide microspheres having a concentration of 1 wt % and a particle size of 200 nm to 250 nm were added in 2 wt % of 2-hydroxy-4-methoxybenzophenone, wherein 2-hydroxy-4-methoxybenzophenone is a UVA absorber. Then, the porous titanium dioxide microspheres were coated on a UV test paper to form three titanium dioxide thin films T. Then, the UV test paper was continuously irradiated by UV for 2 minute to measure the absorption spectrum of the

UV test paper. The results thereof are as shown in FIG. 6A. The UV test paper was left to stand for 20 minutes prior to 2 minutes of continuous UV irradiation, then to measure the absorption spectrum thereof. The results thereof are as shown in FIG. 6B.

### COMPARATIVE EXAMPLE 13

The forming method of comparative example 13 is similar to that of experimental example 8, and the difference thereof is that in comparative example 13, a commercial titanium dioxide was used to form three titanium dioxide thin films P to perform a UV test paper test. Specifically, in comparative example 13, the degree of coloration of the UV test paper was measured by adding 2 wt % of 2-hydroxy-4-methoxybenzophenone in titanium dioxide having a concentration of 1 wt % (made by Parsol). The results thereof are as shown in FIG. 6A. The UV test paper was left to stand for 20 minutes prior to 2 minutes of continuous UV irradiation, then to measure the absorption spectrum thereof. The results thereof are as shown in FIG. 6B.

As shown in FIG. 6A, since the degree of coloration of the titanium dioxide stacked structure TPP of experimental example 4a is less, the effect of UV absorption or blocking thereof is better. In comparison to experimental example 8 and comparative example 13, the titanium dioxide stacked structure TPP of experimental example 4a has greater UV protection capability and can provide an additional 15% to 20% of UV protection capability. In other words, the porous titanium dioxide microspheres of the invention can enhance the effect of UV absorption of a chemical UV absorber. It can be known from FIG. 6B that, in comparison to experimental example 8 and comparative example 13, the titanium dioxide stacked structure TPP of experimental example 4 still has greater UV protection capability after being left to stand for 20 minutes.

Moreover, it can be known from FIG. 5, FIG. 6A, and FIG. 6B that, by using the porous titanium dioxide microspheres of the invention as a bottom layer and further including other commercial sun protection products (containing physical sun protection products or chemical sun protection products), the UV protection capability of the other commercial sun protection products can be enhanced.

### EXPERIMENTAL EXAMPLE 9

A sun protection factor (SPF) test and a protection of UVA (PA) test were performed on 5 wt %, 10 wt %, and 15 wt % of the porous titanium dioxide microspheres having a particle size of 200 nm to 250 nm synthesized in an autoclave by using a self-sacrificing template method. The results thereof are as shown in Table 1 and Table 2.

### COMPARATIVE EXAMPLE 14

A sun protection factor (SPF) test and a protection of UVA (PA) test were performed on a commercial titanium dioxide (product of Merck, model: UV TITAN M160) in concentrations of 5 wt %, 10 wt %, and 15 wt %. The results thereof are as shown in Table 1 and Table 2.

TABLE 1

Sun protection factor (SPF)

| | Experimental example | Comparative |
|---|---|---|
| Concentration | 9 | example 14 |
| 5 wt % | 8 | 5 |
| 10 wt % | 16 | 10 |
| 15 wt % | — | 20 |

It can be known from Table 1 that, in 5 wt % and 10 wt % concentrations, the SPF value of the porous titanium dioxide microspheres of experimental example 9 is greater than the SPF value of the commercial titanium dioxide of comparative example 14. It can therefore be known that, the titanium dioxide having a particle size of 200 nm to 250 nm of experimental example 9 has better protection capability against UV of UVB wave band.

Table 2 lists the PA values after a protection of UVA (PA) test of 5 wt % of experimental example 9 and comparative example 14, wherein a greater PA value represents better UVA protection capability.

TABLE 2

Protection of UVA (PA)

| | Experimental example | Comparative |
|---|---|---|
| Concentration | 9 | example 14 |
| 5 wt % | 5 | 5 |

It can be known from Table 2 that, the sun protection factor of the porous titanium dioxide microspheres having a particle size of 200 nm to 250 nm of experimental example 9 is the same as that of the commercial titanium dioxide of comparative example 14 for UV of UVA wave band. In other words, the protection capabilities of the two are similar.

Based on the above, the sun protection composition of the invention has porous titanium dioxide microspheres having a particle size of 100 nm to 300 nm, such that the sun protection composition can scatter light in a wavelength range between 200 nm and 400 nm. Therefore, the cosmetic and the fabric of the invention containing the sun protection composition can also scatter light in a wavelength range between 200 nm and 400 nm, such that the UV protection capability of the cosmetic and the fabric is enhanced. Moreover, by using the sun protection composition of the invention as a bottom layer and further including other commercial sun protection products, the UV protection capability of the other commercial sun protection products can be enhanced.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

1. A sun protection composition, comprising:
a UV absorber absorbing a light of at least one of UVA radiation and UVB radiation; and
a plurality of porous titanium dioxide microspheres having a particle size of 100 nm to 300 nm, and the porous titanium dioxide microspheres scatter a light in a wavelength range between 200 nm and 400 nm.

2. The sun protection composition of claim 1, wherein based on 100 wt % of the sun protection composition, a content of the porous titanium dioxide microspheres is 1 wt % to 15 wt % and a content of the UV absorber is less than 15 wt %.

3. The sun protection composition of claim 1, wherein the UV absorber comprises: a component (A), a component (B), or a combination thereof, wherein:
the component (A) comprises: avobenzone, oxybenzone, terephthalylidene dicamphor sulfonic acid, or a combination thereof; and
the component (B) comprises: octyl methoxycinnamate, octocrylene, salicylate, or a combination thereof.

4. The sun protection composition of claim 3, wherein based on 100 wt % of the UV absorber, a content of the component (A) is less than 15 wt % and a content of the component (B) is less than 15 wt %.

5. The sun protection composition of claim 1, wherein a ratio of a long diameter and a short diameter of every porous titanium dioxide microsphere is between 0.5 and 1.5.

6. The sun protection composition of claim 1, wherein a difference of any two diameters of the porous titanium dioxide microspheres is less than 50 nm.

7. The sun protection composition of claim 1, wherein a particle size distribution of the porous titanium dioxide microspheres is less than 20%.

8. The sun protection composition of claim 1, further comprising a plurality of ultrafine titanium dioxide spheres, wherein a particle size of the ultrafine titanium dioxide spheres is less than a particle size of the porous titanium dioxide microspheres.

9. The sun protection composition of claim 8, wherein the ultrafine titanium dioxide spheres, the porous titanium dioxide microspheres, and the UV absorber are mixed to form a single agent.

10. A sun protection set, comprising the sun protection composition of claim 8, wherein the porous titanium dioxide microspheres and the UV absorber are mixed to form a first agent, and the ultrafine titanium dioxide spheres form a second agent.

11. A method of using the sun protection set of claim 10, comprising:
forming a first sun protection layer using the first agent; and
forming a second sun protection layer using the second agent to cover the first sun protection layer.

12. The method of claim 11, wherein the second sun protection layer comprises one sun protection layer, two sun protection layers, or a plurality of sun protection layers.

13. A cosmetic having sun protection efficacy, comprising the sun protection composition of claim 1, wherein the cosmetic is an emulsion, a cream, a suspension, a gel, a powder, or a combination thereof.

14. The cosmetic having sun protection efficacy of claim 13, wherein when the cosmetic is an emulsion, a cream, a suspension, or a gel, the cosmetic further comprises:
a carrier oil, an emulsifier, an antibacterial agent, a humectant, and a solvent.

15. A fabric having sun protection efficacy, comprising the sun protection composition of claim 1, wherein the sun protection composition covers a surface of a substrate or is mixed in the substrate.
